(19) United States
(12) Patent Application Publication
(10) Pub. No.: US 2016/0022952 A1
(43) Pub. Date: Jan. 28, 2016

(54) Apparatus and Method for Adaptive Closed-loop Control of Oxygen-Hemoglobin Saturation Levels

(71) Applicant: NAB Medical, Inc., Winchester, MA (US)

(72) Inventor: Douglas A. Brown, Wilmington, MA (US)

(73) Assignee: NAB Medical, Inc., Winchester, MA (US)

(21) Appl. No.: 14/803,171

(22) Filed: Jul. 20, 2015

Related U.S. Application Data
(60) Provisional application No. 62/027,598, filed on Jul. 22, 2014.

(51) Int. Cl. A61M 16/10; A61M 16/06; A61M 16/00; A61M 16/20; A61B 5/145; A61B 5/00

(52) U.S. Cl. CPC A61M 16/1005; A61B 5/14542; A61B 5/7246; A61B 5/7275; A61M 16/0051; A61B 5/7435; A61B 5/7475; A61B 5/0022; A61B 5/7221; A61M 16/202; A61M 16/0627; A61M 16/0672; A61M 2202/0208; A61M 2205/502; A61M 2205/3334; A61M 2016/1025; A61M 2205/3303; A61M 2230/205; A61M 2230/42; A61M 2230/04

(57) ABSTRACT

The present invention is a method, system, and apparatus for providing automated closed-loop control of oxygen-hemoglobin saturation, SₚO₂, levels that adapts to the specific, unique, and variable needs of the individual patient in real-time. A method, system, and standalone breathable gas blending apparatus for controlling SₚO₂ levels in human patients requiring supplemental oxygen therapy, inclusive of adults, pediatrics, and neonates, are described.

### RELATED CASE INFORMATION

This case claims priority benefit from U.S. Provisional Application No. 62/027,598, filed Jul. 22, 2014 entitled Apparatus and Method for Closed-loop Control of Oxygen-hemoglobin Saturation Levels (S_{P}O₂)”, which is incorporated herein by reference in its entirety.

### BACKGROUND

The present invention is in the technical field of medical instrumentation and devices. More specifically, the present invention is in the technical field of oxygen therapy management.

Conventional oxygen therapy comprises a gas delivery system that provides a patient with a blended breathable mixture of oxygen and a carrier gas, usually air. Typical delivery systems include mechanical ventilation, gas confining hoods or masks, and continuous positive airway pressure devices (CPAP). The oxygen fraction of the delivered mixture, often expressed as a percentage, is commonly referred to as the “fraction of inspired oxygen,” and denoted FᵢO₂. Regulation of the FᵢO₂ is used to control the oxygen-hemoglobin saturation (SₚO₂) in the patient.

Presently, standard patient care for adjusting the blend of oxygen and the carrier gas is handled manually by the care provider. An initial FᵢO₂ value is selected and set. Sensors, typically pulse-oximeters, are used to monitor the patient's SₚO₂. Excursions of the SₚO₂ level outside of a prescribed range, defined by minimum and maximum values, trigger an alarm by the SₚO₂ monitor that alerts care providers that a change in FᵢO₂ may be required. The care provider must then assess the situation, estimate the required change in FᵢO₂, and manually adjust the oxygen blend to effect the FᵢO₂ adjustment. This manual procedure places a heavy burden on the care provider, is prone to errors and misadjustments, allows variations in the standard of care arising from variations in expertise of individual care providers, and introduces risk to the patient. Low SₚO₂ levels can produce serious physical and neurological damage, while high levels can be toxic (oxygen toxicity). Further, it is well established that low-birth-weight infants exposed to excessive oxygen can suffer severe eye damage and visual impairment (e.g., retinopathy of prematurity, “ROP”).

Representative prior art includes: (1) U.S. Pat. No. 6,512,938 B2, issued Jan. 28, 2003 to Claure et al.; (2) U.S. Pat. No. 7,802,571 B2, issued Sep. 28, 2010 to Tehrani et al.; and (3) U.S. Publication No. US 2011/0290252 A1, published Dec. 1, 2011 to Amjad et al. These three examples of prior art share several common themes. Each claims control of an external device, Claure a ventilator or air blender, Tehrani a ventilator, and Amjad a servo motor interfacing with a blending knob. Each uses criteria to define a state of the patient for determining the algorithm branch to be used in generating the response signal. Claure uses “counters,” Tehrani employs a “loop indicator,” and Amjad implements a direct state machine approach. All three also utilize a linear response model typical of standard control theory, with Claure and Tehrani invoking PDI (proportional, derivative, integral) methods, and Amjad applying a more complex Kalman Filter estimation system. Each of these disclosures is fundamentally limited by the linear response model and standard control theory approach. In developing the present invention, it has been recognized that oxygen therapy in humans, especially highly-variable neonates, is better managed by non-linear response models. Moreover, implementation of any of the approaches in the three prior disclosures requires additional hardware and software that must be customized to interface with standard respiratory equipment.

### SUMMARY

The current invention improves on the prior art by providing a non-linear, empirical response model for the delivery of a blended breathable mixture of oxygen and a carrier gas, usually air, to a patient. This model was developed based on data collected during extensive human neonatal trials. Building on the results and observations from these trials, the model deviates substantially from traditional control theory. Generally, it was found that specific relationships exist between the current heart rate, current FᵢO₂, current SₚO₂, the recent trend of each, the current trend of each, and the observed SₚO₂ in the next measurement cycle. More specifically, these parameters and the associated trends can be combined into non-linear mathematical expressions that predict the future SₚO₂ and calculate adjustments to the current FᵢO₂ that are needed to maintain the SₚO₂ within the prescribed range. More specifically still, the cited trends are combined with the current FᵢO₂ and SₚO₂ values to compute adjustment parameters that are then inserted into the non-linear expressions to calculate a final FᵢO₂ set point. The result is an approach that continually adapts the blended mixture to appropriate levels for different patients based on the measured ongoing levels within individual patients, and improves the consistency of maintaining hemoglobin oxygen saturation levels within a prescribed range.

Additionally, the current invention includes a standalone blending apparatus and method for blending that can interface seamlessly with existing respiratory medical equipment. The design is compact, suitable for attachment to mobile intravenous (“IV”) stands or direct placement on or adjacent to standard medical respiratory equipment such as ventilators, hood delivery systems, isolettes, CPAP, etc. The invention comprises inlets for oxygen and a breathable blend gas (typically air), a compact manifold with control valves (optimized to minimize system response time), a microprocessor, controller electronics, communication electronics, flow sensors, oxygen sensors, data storage, a graphical user interface and a single outlet to deliver a blended oxygen mixture at the computed FᵢO₂ set point. Power is provided by standard electrical outlets or by a battery. Connection to the patient and existing medical equipment is simple and straightforward. Oxygen and breathable blend gas lines normally attached to the respiratory gas delivery system are instead attached to the apparatus of this invention. The outlet of this apparatus is connected to either inlet (most suitably the oxygen inlet) of the gas delivery system. The gas delivery system is then adjusted to accept 100% of the flow from the inlet to which the apparatus is attached. The blended mixture from the apparatus is then delivered to the patient in the manner prescribed by the respiratory gas delivery system. Response and feedback from the patient is obtained directly from the standard patient monitoring systems, typically a pulse oximeter, with the monitoring systems' output signals delivered directly to the apparatus of this invention.

The method and apparatus of this invention provide a closed-loop system that is effective, easily implemented, eliminates the need for continuous manual adjustment (which is the current standard method), and provides superior care for the patient.

### BRIEF DESCRIPTION OF THE DRAWINGS

For a better understanding of the present invention, and to describe its operation, reference will now be made, by way of example, to the accompanying drawings. The drawings show the different components of the present invention in which:

FIG. 1 is a block diagram depicting one embodiment of the patient oxygen therapy system of the present invention;

FIG. 2 is a diagram depicting the input/output signals, the relevant electronics and the interconnections between them;

FIG. 3 is a block diagram schematic of a gas delivery system or blender; and

FIG. 4 is a flowchart summarizing the logic used to predict and control the FᵢO₂ and thereby the patient's SₚO₂.

### DETAILED DESCRIPTION OF THE INVENTION

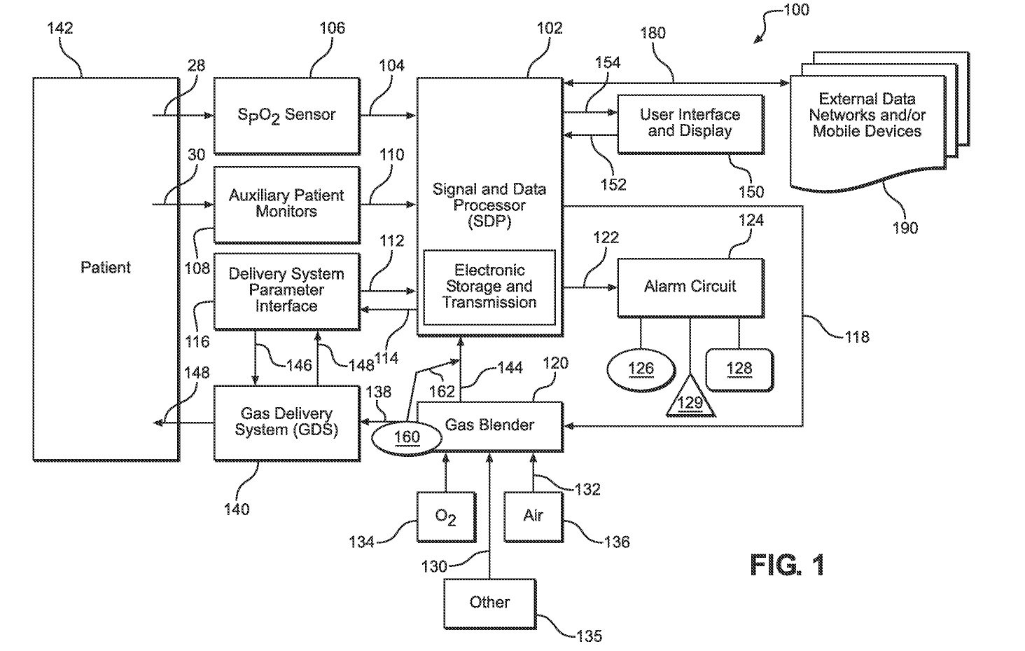

The present invention will now be described more fully with reference to the accompanying drawings. It should be understood that the invention may be embodied in many different forms and should not be construed as limited to the embodiments set forth herein. Throughout the FIGS. 1-4, like elements of the invention are referred to by the same reference numerals for consistency purposes.

Referring now to the invention in more detail, FIG. 1 displays a block diagram depicting an embodiment of a patient oxygen therapy system 100. A signal and data processor (SDP) 102 controls the system and comprises electronic circuitry including but not limited to analog-to-digital conversion (A/D), a central processing unit (CPU), data storage, and a graphical user interface (GUI). SDP 102 receives signals 104 representing the level of oxygen-hemoglobin saturation from an SₚO₂ sensor 106. One or more auxiliary patient monitors 108 provide additional signals 110 to SDP 102, which may include but are not limited to a patient's vital signals such as pulse rate and respiration. These additional signals 110 may be used by SDP 102 to validate incoming data streams to SDP 102, learn patient responses and trends, and monitor critical patient parameters. Additionally, other interface signals 112 and 114 are exchanged between SDP 102 and a delivery system parameter interface 116, wirelessly or by direct connection, and may include but are not limited to mechanical respiration rate, volumetric gas flow, gas pressures, and oxygen concentration.

Post data processing and analysis, SDP 102 issues a command stream 118 to a gas blender 120. The command stream 118 includes but is not limited to an FᵢO₂ set point command generated based on a number of measurements and used to set a blended oxygen mixture 138 to the computed concentration. Blended mixture 138 is then delivered to a gas delivery system (GDS) 140. If SDP 102 determines that the patient or system or an element thereof is in a state of a prescribed alarm condition such as persistent desaturation of the patient's hemoglobin, electrical power loss, gas flow disruption, or other care provider specified condition, an alarm signal 122 is sent to an alarm circuit 124. Alarm circuit 124 then alerts the care providers of the alarm condition via audible, visual or tactile means, such as for example using a speaker 126, a screen 128 and/or a vibration element 129 that is touch sensitive. Alarm circuit 124 may be placed in close proximity to the patient, or in a remote location from the patient such as at a nurse's station for a particular hospital unit or area covered by a care provider. Alarm signal 122 may be transmitted through wires or wirelessly by conventionally available means used for wireless computer/data networks or communications with mobile electronic devices such as cellular telephones and computer tablets.

GDS 140, which may comprise mechanical ventilation, flow rate controllers, continuous positive airway pressure systems (CPAP), nasal cannula, gas confining hoods, masks, and other known components, receives a flow of blended gas with the SDP-determined FᵢO₂ from gas blender 120 over blended gas flow line 138. GDS 140 is of a type that is known, typical examples of which are: the Infant Star, Newport, and Puritan Bennett series of ventilators, and CPAP machines offered by a number of companies.

A delivery system parameter interface 116, which may be independent from or integrated within GDS 140, provides relevant and appropriate parameters from SDP 102 on input 114 for controlling GDS 140 via delivery system parameter interface 116. These parameters may include but are not limited to ventilation rate, inspiratory and expiratory pressures, pulsed flow volumes, flow rates, and continuous flow pressures. Current values of this plurality of parameters are provided to SDP 102 via signal 112 for validation and verification. These parameters are exchanged between delivery system parameter interface 116 and GDS 140 via a plurality of signals 146 and 148, and may be adjusted to set points prescribed by the attending care provider, determined by on-board logic within GDS 140, or by SDP 102.

GDS 140 then delivers a blended gas flow 148 to patient 142 with the FᵢO₂ determined and set by SDP 102, comprising the proper delivery parameters as set within GDS 140.

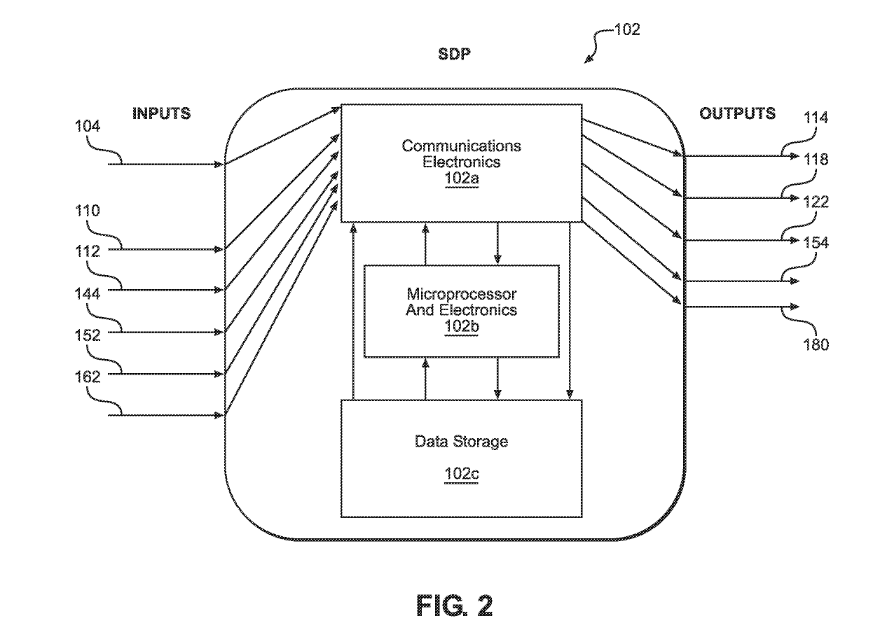

FIG. 2 is a detailed diagram of SDP 102. The key architectural blocks comprising SDP 102 are a communication electronics block 102a, microprocessor and electronics block 102b, and data storage block 102c. Inputs are shown entering SDP 102 from the left, with outputs of SDP 102 exiting on the right. Essential inputs include patient SₚO₂ data 104 from SₚO₂ sensor 106, blender feedback 144 from oxygen sensor 160, and user interface communications 152 from user interface 150. Additional inputs may include but are not limited to auxiliary patient monitors (such as EKG and respiratory data streams) 110 from auxiliary patient monitors 108, parameters from GDS 140 via a delivery system parameter interface 112, which might include real time pressure settings and flow demand, and an auxiliary oxygen concentration sensor signal 162 from oxygen sensor 160. Required output signals include command streams 118 to gas blender 120, alarm states 122 to alarm circuit 124 and data streams and responses 154 to the user interface 150. Additional outputs might include but are not limited to data signals 114 to delivery system parameter interface 116. Examples of these data signals 114 would be status (e.g., pressure, flow, FᵢO₂ set point and other settings, as well as specific patient data from SₚO₂ sensor 106 and auxiliary patient monitors 108) and command strings output to GDS 140 directing GDS 140 to use prescribed settings such as valve openings, flow, pressure, etc. Finally, a direct communication path for sending signals 180 to external data networks and/or mobile devices 190, which may include external computers, networks, mobile phones, tablets, and other devices, may be provided by the communications electronics block 102a either wired or wirelessly. In this embodiment of SDP 102, executable programs, including the FᵢO₂ set point computation, are resident within the microprocessor and electronics block 102b, with the user interface being responsible for data input, output, and display. Other embodiments might have these functions and responsibilities integrated into a single architectural block.

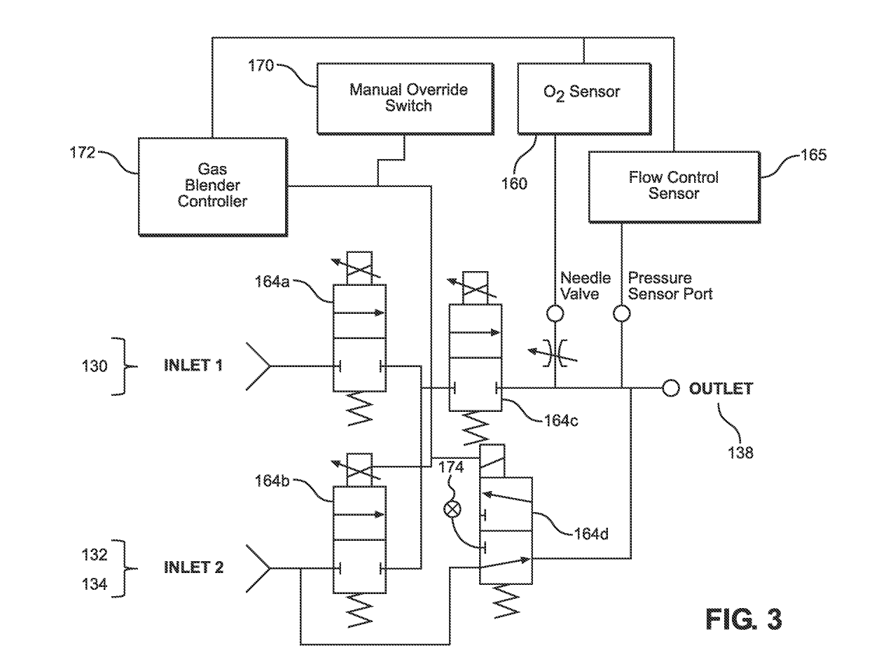

Specifically, in this embodiment, microprocessor and electronics block 102b uses the patient heart rate, current SₚO₂, current FᵢO₂, and trends associated with each to predict the patient SₚO₂ at the next measurement cycle and from this collection of values, combined with the care provider specified SₚO₂ range, compute an FᵢO₂ set point. This set point is input to software resident in microprocessor and electronics block 102b. This software uses feedback from oxygen sensor 160 to provide closed loop control of the oxygen concentration, maintaining it at the determined set point. With this specific valve arrangement, it may be possible for the outlet pressure and flow to vary as a function of FᵢO₂ set point. Control is based on receipt of a signal from a flow control sensor 165 (see FIG. 3), which may sense pressure, flow, or both, and, in concert with valves 164 (see FIG. 3), ensures constant pressure and flow at outlet 138.

FIG. 3 is a detailed diagram of a representative embodiment of gas blender 120 which may be comprised of controllers, valves, regulators, pressure sensors and other components that are known to be incorporated in a gas blender. In the embodiment of FIG. 3, four valves 164a-d are shown. Valves 164a, 164b, and 164c are in a closed position at the start of operation of gas blender 120. During operation, a gas blender controller 172 sets valves 164a and 164b to an open position to permit appropriate flow in accordance with the computed set point FᵢO₂. The position of valve 164c is set to maintain a constant flow and pressure at outlet 138. Valve 164d is closed when energized, blocking the flow during standard operation as denoted by a circled “x” symbol 174 in FIG. 3. Valve 164c is open when de-energized and is used as a fail-safe valve in the event of complete power loss or manual intervention by the care provider. If power to the unit is completely disrupted, or should the care provider decide to intervene manually, a manual intervention switch 170 is used to override control by gas blender controller 172 and valves 164a, 164b, and 164c will close. Valve 164d will open to provide the gas stream from inlet 2 to be delivered through outlet 138 to GDS 140. Selection of the gas to be provided at inlet 1 or inlet 2 is specified by the care provider and determines which gas, oxygen or the breathable blend gas, is to be delivered to outlet 138 under fail-safe or manual intervention conditions.

Under normal operation, gas blender 120 receives a flow of oxygen 130 at inlet 1 and a carrier gas 132 at inlet 2, typically air from external sources 132 respectively. As an alternative to the blending of oxygen 130 and carrier gas 132 by gas blender 120, another breathable gas 134, such as nitrogen for example, could be input to gas blender 120 and used as a blending gas. Oxygen is then blended with the carrier gas to the concentration (FᵢO₂) computed by SDP 102. An auxiliary oxygen concentration sensor 160 is also provided within gas blender 120 or, alternatively, in line with the blended gas flow output line 138 from gas blender 120, to monitor the actual FᵢO₂ flowing from gas blender 120 through a gas delivery system 140 and then delivered to patient 142. Signals 162 from the auxiliary oxygen sensor 160 are provided directly to SDP 102 on sensor line 144 for verification.

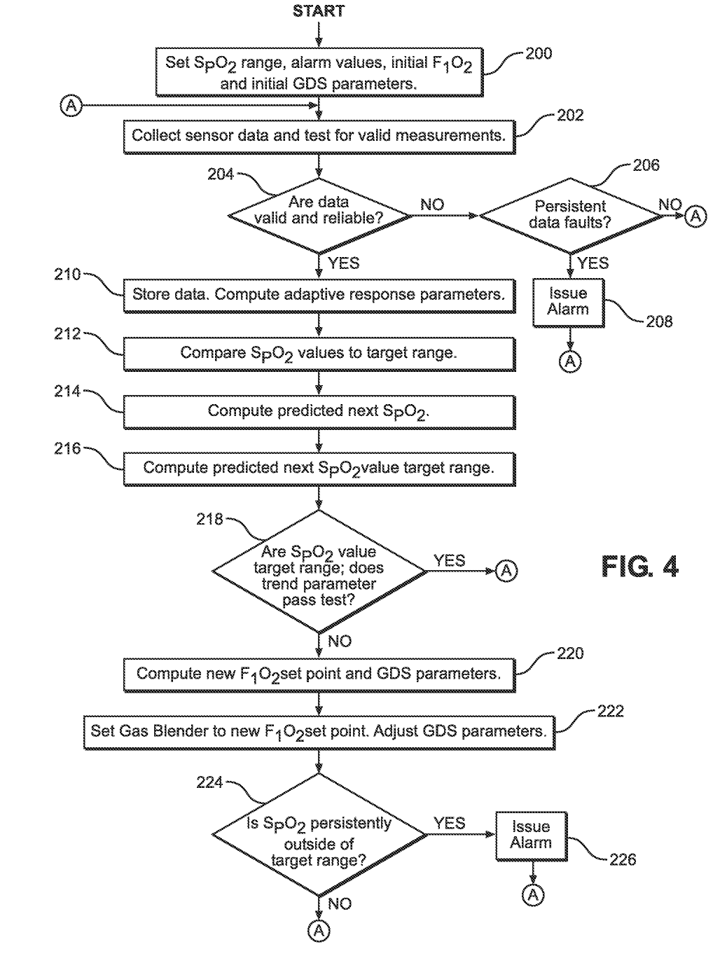

Referring now to the logic implemented in SDP 102, FIG. 4 presents a flow chart for controlling an appropriate blend of gas delivered to patient 142 by system 100. As shown in the diagram, the process begins at Step 200 by storing the specified target SₚO₂, the specified acceptable SₚO₂ range and the specified alarm values (e.g., SₚO₂ alarm window and heart rate thresholds), then initializing the system to the current FᵢO₂ and other possible input parameters that might include GDS values such as pressure, flow, and respiration rate. Of these, the most critical inputs are the SₚO₂ target and range. These values are determined by the attending medical staff or care provider and follow guidelines established by the medical standards and protocols in use at the specific institution or by the individual care provider. While the actual numbers may vary slightly by provider, a typical target and range, for example, may be a target hemoglobin oxygen saturation level (SₚO₂) of 96% with an allowable low of 94% and a maximum of 98%. Such a range would generally ensure that the patient is exposed neither to hypoxic nor hyperoxic conditions.

In step 202, the main loop begins during which sensor data are collected, stored, and validated. Tests in step 202 may include, but are not limited to, a comparison of pulse rates as measured by auxiliary patient monitors 108 such as a pulse-oximeter, an independent electrocardiogram (EKG) and/or other monitors that may be connected to the system to provide real-time or historical patient data. If the differential between compared rates differs within a prescribed amount, it is determined whether the data are accepted as valid and reliable at step 204. This test at step 204 serves to reject invalid data affected by motion artifact or other sensor disturbance at the patient. If the data persistently test as invalid, it is determined whether the invalid data is indicative of a sensor failure or fault, such as a loose connection at step 206. If so, an alarm is issued at step 208 and the flow returns to step 202.

Valid data at step 204 are gathered and used to compute adaptive response parameters (“ARPs”). In turn, these ARPs, also referred to as sensitivity scalars, are stored in a vector array, with each parameter deriving from historical trends presented by the individual patient at step 210. Gathering and analyzing data related to the specific relationships between the current heart rate, current FᵢO₂, current SₚO₂, recent trend of each, current trend of each, and the observed SₚO₂ in the next measurement cycle provides one or more bases to generate the ARPs. These scalars would be used initially to compensate for variations in response times for specific GDS models, set and forget. They could also be used for especially difficult patients with anomalous responses, rare, but can occur. In addition, the response is designed to address concerns including the immediate state of the patient, (e.g., severe drop in pulse or SₚO₂) as well as the time for the patient to respond (e.g., the patient is marginally outside of the target window but due to fluid accumulation in the lungs is not responding in a normal or timely manner).

If the data at step 204 is determined to be valid, step 210 is executed and the flow continues to step 212 where the actual value of the SₚO₂ as reported by sensor 106 is compared to the target range as prescribed and input by the attending care provider. The position of the actual value with respect to the midpoint of the range is used in the calculation of FᵢO₂ adjustments and to test for alarm conditions. For example, FᵢO₂ may be set in a range (e.g., 0-100% or room air, 21-100%).

In Step 214, the predicted next measured SₚO₂ value is based on the patient's history-based ARPs. Analysis of actual patient histories and responses reveals relationships between the identified ARPs, the current FᵢO₂, the current SₚO₂, and the future SₚO₂. Moreover, these relationships, when expressed in a non-linear mathematical statement, can be used to compute the FᵢO₂ setting required to maintain the patient's SₚO₂ near the specified target and within the specified range.

The predicted SₚO₂ values are compared to the target and range in Step 216. If the test criteria of step 218 are not satisfied, a new FᵢO₂ set point and new GDS parameters are computed at step 220, and gas blender 120 and GDS 140 are adjusted appropriately at step 222. If the test criteria at step 218 are met, the flow is returned to the top of the main loop at step 202 and the process is repeated.

In the event that a persistent SₚO₂ excursion outside of the target range is noted at step 224, an alarm is issued at step 226 to notify the attending care provider by audible and/or visual means on speaker 126 and/or on screen 128. Following execution of step 224 or step 226, the flow is returned to the top of the main loop at step 202 and the process is repeated.

The advantages of the present invention include, without limitation, provision of an automated device and ways to control the FᵢO₂ and other parameters for patients receiving supplemental oxygen therapy. By automating the control of these parameters, especially FᵢO₂, regulation of SₚO₂ is improved, human error is reduced or eliminated, patient safety is enhanced, and superior clinical outcomes can be realized, especially in low-birth-weight neonates (e.g., reduction in incidence and/or severity of retinopathy of prematurity).

In one embodiment of the present invention, the SDP 102, alarm circuit 124, and gas blender 120, are combined into a single compact and portable package. This package would provide a single unit that could be readily transported. This embodiment may also be constructed to provide a flexible electro-mechanical interface suitable for connection to and communication with any combination of sensors, monitors, parameter interfaces, and gas delivery systems. Advantages of this embodiment of the present invention include, without limitation, that the control package is separable, may be readily moved among multiple oxygen therapy configurations (e.g., from a mechanical ventilator to CPAP or other delivery means), and is readily transportable with or by the patient.

In a specific embodiment of SDP 102, data collection, storage, and transmission devices would be incorporated to provide on-demand and/or continuous streams of data accessible by fixed and mobile devices, including but not limited to computers, tablets, smart phones and other similar devices. Historical archives of data would also reside within SDP 102 for at-will access by users and other authorized parties.

Connection to the patient and existing medical equipment is simple and straightforward. Oxygen and breathable blend gas lines normally attached to GDS 140 are instead attached to the apparatus of this invention. The outlet of this apparatus is connected to either inlet (most suitably the oxygen inlet) of GDS 140. The gas delivery system is then adjusted to accept 100% of the flow from the inlet to which the apparatus is attached. The blended mixture from the apparatus is then delivered to the patient in the manner prescribed by the respiratory gas delivery system. Response and feedback from the patient is obtained directly from the standard patient monitoring systems, typically a pulse oximeter, with the monitoring systems' output signals delivered directly to the apparatus of this invention.

The present invention is a method, system, and apparatus for providing closed-loop control of oxygen-hemoglobin saturation levels, SₚO₂, that adapts to the specific, unique, and variable needs of the individual patient in real-time. While the invention has been described with respect to the figures, it will be appreciated that many modifications and changes may be made by those skilled in the art without departing from the spirit of the invention. Any variation and derivation from the above description and drawings are included in the scope of the present invention as defined by the claims.

What is claimed is:

1. A method for adjusting and blending inspired oxygen delivered to a patient in response to a hemoglobin oxygen saturation level measured in the patient, comprising:
(a) specifying a target range of hemoglobin oxygen saturation levels through a user interface in communication with a signal and data processor (SDP);
(b) specifying an initial inspired oxygen level to be delivered to the patient as determined by the care provider through the user interface;
(c) measuring a hemoglobin oxygen saturation level of a patient at an SₚO₂ sensor;
(d) transmitting a signal with a measured hemoglobin oxygen saturation value of the patient from the SₚO₂ sensor;
(e) receiving the signal from the SₚO₂ sensor at the SDP;
(f) comparing the measured hemoglobin oxygen saturation value to at least one preset patient parameter and a target rate of change of the at least one preset patient parameter using the SDP;
(g) determining whether the measured hemoglobin oxygen saturation value is within the target range using the SDP;
(h) incorporating the difference between the target and measured oxygen hemoglobin values into an adaptive response parameter;
(i) computing the hemoglobin oxygen saturation value predicted to occur at the next measurement;
(j) comparing the predicted hemoglobin oxygen saturation value to the target value and range, and predicted future values of the patient's hemoglobin saturation level;
(k) if it is determined that the predicted hemoglobin oxygen saturation value is outside the target range using the SDP, determining a significant adjustment factor using the SDP to be applied to the inspired oxygen level delivered to the patient based on the current valid measurement, specific patient trends, and predicted future values of the patient's hemoglobin saturation level;
(l) if it is determined that the predicted hemoglobin oxygen saturation value is within the target range using the SDP, determining a slight adjustment factor using the SDP to be applied to the inspired oxygen level delivered to the patient based on the current valid measurement, specific patient trends, and predicted future values of the patient's hemoglobin saturation level;
(m) transmitting a signal from the SDP to a gas blender that controls the inspired oxygen level delivered to the patient to adjust the inspired oxygen level according to the significant adjustment factor or the slight adjustment factor; and
(n) transmitting a signal from the SDP to an alarm circuit causing an alarm to be signaled in the event that the significant factor is applied to adjust the inspired oxygen level.

2. The method of claim 1, wherein the significant adjustment factor and the slight adjustment factor are applied to the inspired oxygen level to be delivered to the patient to maintain the patient's hemoglobin saturation value within the target range.

3. The method of claim 2, wherein the signal from the SDP to the alarm circuit is transmitted after a preset number of applications of the significant factor has failed to return the SₚO₂ to within the prescribed range.

4. The method of claim 1, wherein the adjustment factor is repeatedly re-evaluated and applied, and the alarm to be signaled by continuously repeating steps c-n.

5. The method of claim 1, wherein a breathable gas is combined with pure oxygen to set the inspired oxygen level delivered to the patient, inclusive of 0% oxygen blends.

6. The method of claim 1, wherein the user interface includes a visual display enabling an operator to view a plurality of parameters including at least one from among a group comprising: (a) pressure, (b) flow, (c) respiration rate, (d) pulse rate, (e) measured SₚO₂, (f) predicted SₚO₂, (g) measured FᵢO₂, (h) set point FᵢO₂, and (i) the historical trends of each.

7. The method of claim 6, wherein the user interface and display are used to view and update one or more of a plurality of settings comprising (a) target SₚO₂, (b) SₚO₂ range, (c) FᵢO₂ range, and (d) sensitivity scalars for each ARP.

8. The method of claim 1, wherein the alarm circuit has settings comprising (a) threshold values for pulse rate, (b) threshold values for measured SₚO₂, (c) persistence of pulse rate out of range, and (d) persistence of measured SₚO₂ out of range that may be selected and entered using the user interface.

9. The method of claim 1, wherein the user interface is used to select a mode of operation from a group comprising: (a) fully automatic control; or (b) manual control.

10. A system for controlling the resulting hemoglobin oxygen saturation value of a patient comprising:
(a) an oxygen saturation sensor for measuring a patient's hemoglobin oxygen saturation level;
(b) a user interface and display configured to display measured hemoglobin oxygen saturation levels and patient cardio-respiratory parameters, and inserting and utilizing user inputs of control and calculation parameters;
(c) a transmitter configured to convert the patient's measured hemoglobin oxygen saturation level into an electrical or electromagnetic data signal for transmission to a receiving device such as a wireless computer, computer network, or mobile electronic device;
(d) one or more auxiliary patient monitors configured to monitor patient cardio-respiratory parameters;
(e) a transmitter for converting other patient cardio-respiratory parameters into an electrical or electromagnetic data signal for transmission to a receiving device;
(f) a signal and data processor (SDP) configured to receive and process electrically or electromagnetically transmitted data signals, and providing output indicative of:
i. validity of the input signal;
ii. historical trends of the patient's hemoglobin oxygen saturation levels;
iii. historical trends of other patient cardio-respiratory parameters;
iv. predictions of future patient hemoglobin oxygen saturation levels;
v. patient specific adaptations based on historical trends, current values, and predicted values of patient hemoglobin oxygen saturation levels; and
vi. the fraction of inspired oxygen to be delivered to the patient that will maintain the patient's hemoglobin oxygen saturation level within a prescribed range;
(g) electronic storage, processing, and transmission components of the SDP configured to store, display, and transmit data received or processed;
(h) a gas blender in communication with the SDP and configured to receive breathable gas at two or more inlets, blend gasses input to the inlets and distribute a blended gas through an outlet; and
(i) a gas delivery system receiving the blended gas and delivering the blended gas to a patient.

11. The system of claim 10, wherein the auxiliary patient monitors comprise a pulse-oximeter.

12. The system of claim 10, wherein the SDP comprises a programmable microcomputer.

13. The system of claim 10, wherein the gas blender comprises a plurality of valves that are regulated by a at least one flow controller, at least one pressure controller, at least one sensor, and a gas blend processor connected to each of the valves, flow controllers, pressure controller and sensor.

14. The system of claim 13, wherein the sensor comprises an oxygen sensor configured to monitor oxygen concentration in the blended gas.

15. The system of claim 10, wherein the gas delivery system further comprises a component configured to deliver breathable gas mixtures to a patient of a type from the group comprising: ventilators, hoods, isolettes, nasal cannula, masks, and the like.

16. The system of claim 10, wherein the user interface is used to select a mode of operation from a group comprising: (a) fully automatic control; or (b) manual control.

17. The system of claim 11, further comprising an alarm unit configured to:
display invalid signals and provide an alert; and
issue an alarm upon a system error condition or other preset condition occurring, wherein the alarm is in a form comprising one or more from a group comprising an audible alarm, a visual alarm and/or a tactile alarm.

18. An apparatus for receiving a plurality of breathable gasses, blending and delivering a breathable gas mixture of a controllable composition that maintains hemoglobin oxygen saturation levels within a prescribed range, comprising:
(a) a user interface and display configured to display received data, insert and utilize user inputs of control and calculation parameters;
(b) a signal and data processor (SDP) configured to receive data signals indicating hemoglobin oxygen saturation levels and other cardio-respiratory data, process the data signals and provide an output signal indicating:
i. validity of the input signal
ii. historical trends of the patient's hemoglobin oxygen saturation levels;
iii. historical trends of other patient cardio-respiratory parameters;
iv. predictions of future patient hemoglobin oxygen saturation levels;
v. patient specific adaptations based on historical trends, current values, and predicted values of patient hemoglobin oxygen saturation levels; and
vi. the fraction of inspired oxygen to be delivered to the patient that will maintain the patient's hemoglobin oxygen saturation level within a prescribed range;
(c) a gas blender in communication with the SDP and configured to receive breathable gas at two or more inlets, blend gasses input to the inlets and distribute a blended gas through an outlet; and
(d) a gas delivery system receiving the blended gas and delivering the blended gas to a patient.

19. The apparatus of claim 18, wherein the signal and data processor further comprises one or more of wired and wireless communications components.

20. The apparatus of claim 18, wherein the signal and data processor comprises a human-to-machine interface (HMI) with graphical display and parameter input capability.

21. The apparatus of claim 18, wherein the SDP comprises a programmable microcomputer.

22. The apparatus of claim 18, wherein the device communicates directly with personal electronics, including computers, tablets, cellular phones, smart phones, and the like.

23. The apparatus of claim 18, comprises a plurality of valves that are regulated by a at least one flow controller, at least one pressure controller, at least one sensor, and a gas blend processor connected to each of the valves, flow controllers, pressure controller and sensor.

24. The apparatus of claim 23, wherein the sensor comprises an oxygen sensor configured to monitor oxygen concentration in the blended gas.

25. The apparatus of claim 18, wherein the user interface and display is configured to select either fully automatic (closed loop) control or manual control.

26. The apparatus of claim 18, further comprising an alarm unit:
display invalid signals and provide an alert; and
issue an alarm upon a system error condition or other preset condition occurring, wherein the alarm is in a form comprising one or more from a group comprising an audible alarm, a visual alarm and/or a tactile alarm.
